SEQUENCE LISTING (4 sequences)

SEQ ID NO: 1 - DNA, 60 bp, Artificial Sequence
  Description of Artificial Sequence Synthetic oligonucleotide
atgcccgctagcatgcaccagaatctagatgccacgctagctccgacatgcggcaaccta

SEQ ID NO: 2 - DNA, 11 bp, Artificial Sequence
  Description of Artificial Sequence Synthetic oligonucleotide
atgcccgctag

SEQ ID NO: 3 - DNA, 29 bp, Artificial Sequence
  Description of Artificial Sequence Synthetic oligonucleotide
catgcaccagaatctagatgccacgctag

SEQ ID NO: 4 - DNA, 20 bp, Artificial Sequence
  Description of Artificial Sequence Synthetic oligonucleotide
ctccgacatgcggcaaccta

(19) United States
(12) Patent Application Publication
(10) Pub. No.: US 2021/0110889 A1
(43) Pub. Date: Apr. 15, 2021

(54) GENOME ASSEMBLY METHOD, NON-TRANSITORY COMPUTER READABLE MEDIUM, AND GENOME ASSEMBLY DEVICE

(71) Applicant: HITACHI HIGH-TECHNOLOGIES CORPORATION, Tokyo (JP)

(72) Inventors: Tateo NAGAI, Santa Clara, CA (US); Tsuyoshi OGINO, Tokyo (JP)

(21) Appl. No.: 16/598,319

(22) Filed: Oct. 10, 2019

(51) Int. Cl. G16B 30/20; G16B 20/20

(52) U.S. Cl. CPC G16B 30/20; G16B 20/20

(57) ABSTRACT

Provided is a method of assembling a genome, including: determining the reference appearance rates, that are the appearance rates of all n-base motifs in the nucleotide sequence of a reference genome, in which the n-base motif is a nucleotide sequence containing n bases; and the sample appearance rates, that are the appearance rates of all the n-base motifs in the nucleotide sequences of DNA fragments, calculating the deviations of the sample appearance rates from the reference appearance rates for all the n-base motifs; selecting a predetermined number of n-base motifs having smallest deviations and sample appearance rates of not less than a predetermined value; converting the nucleotide sequences of the DNA fragments into DNA fragments in genome map format using the predetermined number of n-base motifs selected; and assembling the DNA fragments converted in genome map format to generate assemble contigs derived from the DNA in the sample.

### TECHNICAL FIELD

The present invention relates to a method of assembling DNA, a non-transitory computer readable medium, and a device of assembling DNA.

### BACKGROUND ART

DNA holds genetic information of living things, and its analysis is regarded as important. DNA is a polymer of nucleotides each composed of a phosphate, a sugar, and a base. Four types of bases, adenine (A), guanine (G), cytosine (C), and thymine (T), are contained in DNA. The order of nucleotides bound in DNA is called nucleotide sequence. DNA nucleotide sequence is represented as a series of bases (A, G, C, and T), which is a one-dimensional sequence. Since the genetic information in a DNA is held in the form of nucleotide sequence, there is demand for DNA nucleotide sequencing.

The nucleotide sequence of a DNA in a sample is obtained, for example, by preparing fragments of the DNA (hereinafter referred to as “DNA fragment”) from the sample; sequencing the DNA fragments; and assembling (joining) the nucleotide sequences of the obtained DNA fragments. The nucleotide sequence of a DNA fragment can be said to be a part cut from the nucleotide sequence of a DNA. The nucleotide sequence of a DNA fragment is obtained by DNA sequencing. The nucleotide sequence of a DNA fragment obtained by DNA sequencing is, for example, about 10,000-20,000-base (10-20 kb (kilo base)) long on average per single fragment, as an average length with long-read sequencers. In Long Read Sequencing De Novo Assembly, the nucleotide sequences of DNA fragments are assembled based on their overlapping segments (common portions between the nucleotide sequences) to reconstruct the nucleotide sequence of the original DNA.

### PRIOR ART DOCUMENT

### Patent Document

Patent Document 1: Japanese National-Phase Publication (JP-A) No. 2011-514804

### SUMMARY OF THE INVENTION

### Problems to be Solved by the Invention

The nucleotide sequence of a DNA fragment obtained by DNA sequencing, however, may contain errors of about 15%. Errors mean, for example, that an original base is sequenced as another base in DNA sequencing. Thus, Long Read Sequencing De Novo Assembly has difficulty in finding overlapping segments with simple comparison between the nucleotide sequences of DNA fragments. Therefore, consideration of nucleotide sequences similar to each other as overlapping segments is conducted. However, it requires enormous computation amount and memory size to be used to find similar nucleotide sequences from millions of DNA fragments.

The present invention aims to provide a technique for assembling DNA fragments with reduced computation load.

### Means for Solving the Problems

In order to solve the above problem, the following means are used.

In a first aspect, there is provided a method of assembling a genome, the method including allowing a computer to execute the following steps of:

determining the reference appearance rates, that are the appearance rates of all n-base motifs in the nucleotide sequence of a reference genome, wherein the n-base motif is a nucleotide sequence comprising n (n is a predetermined natural number) bases, and wherein the reference genome is the standard genome of an organism; and the sample appearance rates, that are the appearance rates of all of the n-base motifs in the nucleotide sequences of DNA fragments, wherein the DNA fragments is based on DNA extracted from a sample derived from the organism;

calculating the deviations of the sample appearance rates from the reference appearance rates for all of the n-base motifs;

selecting a predetermined number of n-base motifs with smallest deviations, wherein the n-base motifs each have a sample appearance rate of not less than a predetermined value;

converting the nucleotide sequences of the DNA fragments into DNA fragments in genome map format using the predetermined number of n-base motifs selected; and

assembling the DNA fragments converted in genome map format to generate assemble contigs derived from the DNA in the sample.

The aspect of the present disclosure may be implemented by the programs executed by an information processor. Thus, the configuration of the present disclosure may be defined as a program for allowing an information processor to execute the processes implemented by the means in the aspect described above, or a computer readable storage medium storing the program. The configuration of the present disclosure may also be defined as a method for allowing an information processor to execute the processes implemented by the means described above. The configuration of the present disclosure may also be defined as a system including an information processor for executing the processes implemented by the means described above.

### EFFECT OF THE INVENTION

The present invention provides a technique for assembling DNA fragments with reduced computation load.

### BRIEF DESCRIPTION OF THE DRAWINGS

FIG. 1 illustrates an exemplary system configuration of a genome analysis system in embodiments.

FIG. 2 illustrates an exemplary operation flow of the genome assembly device 100.

FIG. 3 illustrates an exemplary palindromic six-base motif.

### DETAILED DESCRIPTION OF THE INVENTION

Embodiments will be described below with reference to the drawings. The configurations of the embodiments are illustrative, and the configuration of the invention is not limited to the specific configurations of the disclosed embodiments. In carrying out the invention, a specific configuration according to the embodiment may be adopted as appropriate.

### EMBODIMENTS

### Examples of Configuration

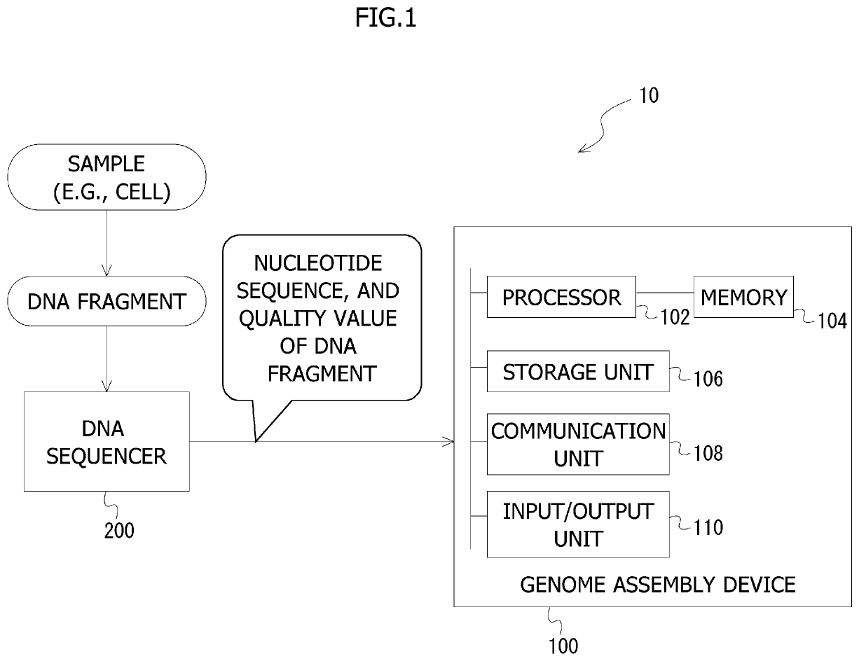

FIG. 1 illustrates an exemplary system configuration of the genome analysis system in the present embodiment. The genome analysis system 10 illustrated in FIG. 1 includes a genome assembly device 100, and a DNA sequencer 200. In the genome analysis system 10, the DNA sequencer 200 receives an input of a DNA fragment extracted from a sample (e.g., cell) from an organism to be analyzed, then reads the nucleotide sequence of the DNA fragment, and output the read nucleotide sequence of the DNA fragment. The genome assembly device 100 converts the nucleotide sequence of the DNA fragment output from the DNA sequencer 200 into genome map format, and then assembles DNA fragments converted to genome map format to produce an assemble contig. In addition, the genome assembly device 100 detects structural variations from the sample, based on the generated assemble contig obtained from the sample and a reference genome converted to genome map format. Although the organism to be analyzed is human in this case, the organism to be analyzed is not restricted to human and may be another animal, a plant, or the like.

The genome assembly device 100 includes a processor 102, a memory 104, a storage unit 106, a communication unit 108, and an input/output unit 110. These are connected to each other by bus. The genome assembly device 100 can be provided by use of a special or general computer, or an electronic device equipped with a computer, such as personal computer (PC), workstation (WS), smartphone, cell phone, or tablet terminal.

The processor 102 loads a program stored in the storage unit 104 or the like into workspace of the memory 104 and runs the program. Through the run of the program, the processor 102 controls the components and the like, enabling the genome assembly device 100 to perform a computation processing and the like. The computation processing performed by the genome assembly device 100 includes processing of converting the nucleotide sequence of a DNA fragment into genome map format; processing of assembling DNA fragments in genome map format to produce an assemble contig; and detecting structural variations of the sample from the assemble contig or the like. The processor 102 is, for example, a central processing unit (CPU) or a digital signal processor (DSP).

The memory 104 stores programs executed by the processor 102, data to be used by the processor 102 to execute programs, and the like. Examples of the memory 104 include random access memory (RAM) and read only memory (ROM).

The storage unit 106 stores various programs executed by the processor 102, and various data and tables to be used by the processor 102. Information to be stored in the storage unit 106 may be stored in the memory 104. Information to be stored in the memory 104 may be stored in the storage unit 106. The storage unit 106 is, for example, an erasable programmable ROM (EPROM), or a hard disk drive (HDD). The storage unit 106 may include removable medium, or portable storage medium. The removable medium is, for example, a universal serial bus (USB) memory, or a disc storage medium, such as a compact disc (CD) or a digital versatile disc (DVD).

The storage unit 106 storages the nucleotide sequences of reference genomes of organisms to be analyzed; the nucleotide sequences of DNA fragments that are output from the DNA sequencer 200; processing programs, for example, for converting the nucleotide sequences of the DNA fragments into a genome map format; and the like.

A genome that serves as a standard of an organism (such as animal or plant) is referred to as reference genome of the organism. A reference genome can be defined in each organism (in each type of animal or the like). The nucleotide sequence of a reference genome is the standard nucleotide sequence of the standard genome. The nucleotide sequence of the human reference genome is present as an about 3 billion-pair nucleotide sequence. A reference genome may be called as, for example, standard genome, standard sequence, referring genome, or referring sequence.

The communication unit 108 is connected to other device, and controls the communication between the genome assembly device 100 and the other device, such as the DNA sequencer 200 and the like. The communication unit 108 is, for example, a local area network (LAN) interface board, a wireless communication circuit for wireless communication, or a communication circuit for wired communication. The LAN interface board or wireless communication circuit is connected to a network such as the Internet.

The input/output unit 110 includes an input device and an output device. The input device includes a keyboard, a pointing device, a wireless remote controller, a touch panel, and the like. The input device may also include a video/image input device, such as a camera, and an acoustic/voice input device, such as a microphone. The output device includes a liquid crystal display (LCD), an electroluminescence (EL) panel, a cathode ray tube (CRT) display, a plasma display panel (PDP), and a printer. The output device may also include an acoustic/voice output device, such as a speaker.

The DNA sequencer 200 is a device for reading the nucleotide sequence of a DNA fragment extracted from a sample (e.g., cell) derived from an organism to be analyzed. The DNA sequencer 200 outputs the read nucleotide sequence of the DNA fragment to the genome assembly device 100. The DNA sequencer 200 herein reads the nucleotide sequences of DNA fragments of about 10,000-20,000-base (10-20 kb) long on average per single fragment. The DNA sequencer 200 also outputs each read base with a quality value added. The DNA sequencer 200 determines the nucleotide sequence based on measured values obtained by a sensor. The sensor determines each base in the nucleotide sequence to be A or T or G or C using various threshold values based on empirical rules. Determining the base is called basecall. The quality of basecall varies depending on the method, and some are not good at an arrangement of the same bases, such as AAAA, and some are not good at a segment having many CG. The quality value is an index representing the reliability of base determination in basecall by a sensor of the DNA sequencer 200.

### Examples of Operation

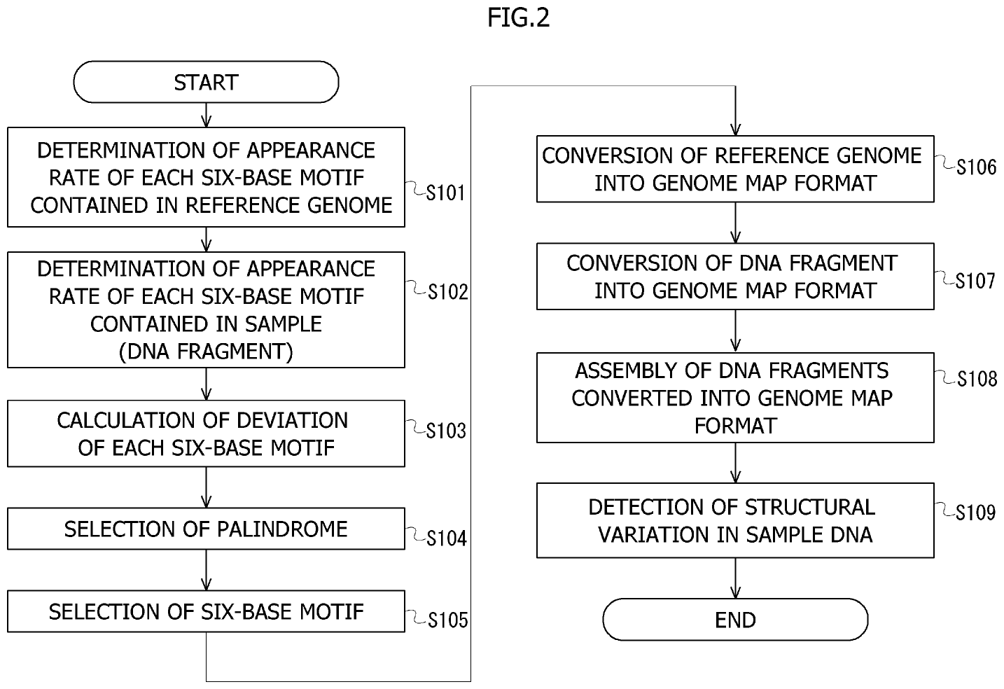

FIG. 2 illustrates an exemplary operation flow of the genome assembly device 100. Before the start of the operation flow illustrated in FIG. 2, the DNA sequencer 200 reads the nucleotide sequence of a DNA fragment extracted from a sample (e.g., cell) derived from an organism to be analyzed, and then outputs the nucleotide sequence read from the DNA fragment and the quality values of the bases to the genome assembly device 100. The genome assembly device 100 receives the nucleotide sequence of the DNA fragment in the sample and the quality values of the bases from the DNA sequencer 200 via the communication unit 108. The genome assembly device 100 stores the received nucleotide sequence of the DNA fragment in the sample and the quality values of the bases in the storage unit 106. The operation flow illustrated in FIG. 2 starts after the genome assembly device 100 obtains the nucleotide sequence of the DNA fragment in the sample and the quality values of the bases from the DNA sequencer 200.

A nucleotide sequence containing a series of n bases (n is a predetermined natural number) is referred to as n-base motif. For example, a nucleotide sequence comprising a series of six bases is a six-base motif. Based on four types of bases, the six-base motif can include 4096 (=4⁶) varieties of six-base motifs. Specifically, the six-base motif is represented, for example, as “ACTTCG” or “CGAATG.”

In S101, the processor 102 of the genome assembly device 100 obtains the nucleotide sequence of a reference genome stored in the storage unit 106. The processor 102 counts the appearance number by which each six-base motif appears in the nucleotide sequence of the reference genome (referred to as reference appearance number). The processor 102 also counts the number of bases contained in the nucleotide sequence of the reference genome (referred to as reference total base number). The reference total base number may be previously stored in the storage unit 106, and the processor 102 may obtain the number of bases contained in the nucleotide sequence of the reference genome from the storage unit 106. Furthermore, the processor 102 calculates the reference appearance rate, the appearance rate of each six-base motif in the reference genome, by dividing the reference appearance number of each six-base motif by the reference total base number. The processor 102 stores the calculated reference appearance number and the reference appearance rate of each six-base motif in the storage unit 106.

In S102, the processor 102 obtains the nucleotide sequences of all DNA fragments and the quality value of each base that have been obtained from the DNA sequencer 200 and stored in the storage unit 106. The processor 102 counts the appearance number by which each six-base motif appears in the nucleotide sequence of all of the DNA fragments (referred to as sample appearance number). The processor 102 also calculates the quality value of each six-base motif based on the quality value of each base. For example, the quality value of each six-base motif is calculated as the average value of the quality values of all bases contained in each six-base motif. The processor 102 also counts the number of bases each contained in the nucleotide sequences of all DNA fragments (referred to as sample total base number). Furthermore, the processor 102 calculates the sample appearance rate, the appearance rate of each six-base motif in DNA fragments in the sample, by dividing the sample appearance number of each six-base motif by the sample total base number. The processor 102 stores the calculated quality value, sample appearance number and sample appearance rate of each six-base motif in the storage unit 106.

In S103, the processor 102 calculates the deviation for each six-base motif. The deviation of a six-base motif is the degree to which the sample appearance rate of the six-base motif deviates from the reference appearance rate of the six-base motif. The deviation is calculated as an absolute value of (1−(sample appearance rate/reference appearance rate)). Smaller deviation means the sample appearance rate closer to the reference appearance rate. The processor 102 stores the calculated deviation of each six-base motif in the storage unit 106. It is considered that there is no significant difference between the reference appearance rate and the actual sample appearance rate of each six-base motif. Thus, it is assumed that smaller deviation means the six-base motif has fewer read errors in the nucleotide sequence by the DNA sequencer 200.

In S104, the processor 102 selects palindromic six-base motifs from the all of the six-base motifs. The term “palindromic” means a structure in which the sequence obtained by reversely reading the complementary strand of a n-base motif (n is a positive even number) is the same as the original n-base motif. When n is an odd number, there is no palindromic n-base motif.

In DNA double helix structure, bases pair with their fixed counterparts. In a DNA double helix, one helix and the other paired helix are complementary strands. For example, when the nucleotide sequence of one helix in a DNA double helix is GTCGAT, the nucleotide sequence of the other helix (complementary strand) is CAGCTA.

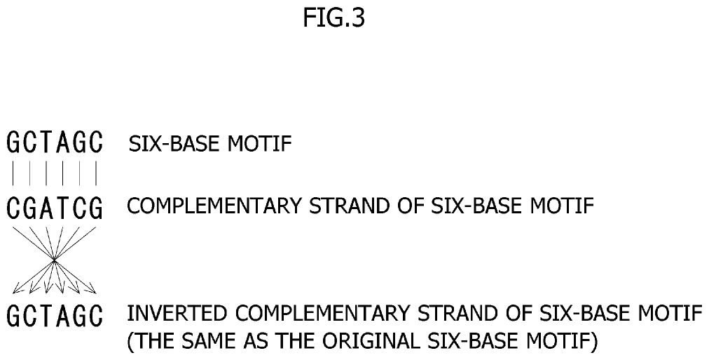

FIG. 3 illustrates an exemplary palindromic six-base motif. The six-base motif “GCTAGC” illustrated in FIG. 3 has a complementary strand of “CGATCG.” When read in the reverse direction (from right to left), the complementary strand of the six-base motif becomes “GCTAGC,” which is the same as the original six-base motif. Such a six-base motif is called palindrome.

In S105, the processor 102 selects six-base motifs to be used in conversion into genome map format. The processor 102 selects six-base motifs, which are the palindromic six-base motifs selected in S104, and have a quality value of the predetermined value or more, and a sample appearance rate of the predetermined value or more. The processor 102 selects four six-base motifs with smallest deviations out of the selected six-base motifs, as the predetermined nucleotide sequences (six-base motifs) to be used in the conversion into genome map format. Although four six-base motifs are selected here, six-base motifs to be selected are not limited to four.

In S106, the processor 102 obtains the nucleotide sequence of a reference genome stored in the storage unit 106. The processor 102 converts the obtained nucleotide sequence of the reference genome into genome map format. The genome map format is a representation format in which bases contained in a nucleotide sequence are classified into bases that match with a predetermined nucleotide sequence (for example, a predetermined n-base motif) and other bases. The predetermined nucleotide sequence is, for example, a nucleotide sequence that serves as an assembly mark when DNA fragments are assembled. In genome map format, for example, the nucleotide sequence to be converted (the nucleotide sequence of the reference genome or DNA fragment) is expressed such that a portion that matches the predetermined nucleotide sequence can be recognized. For example, a predetermined nucleotide sequence (six-base motif) as the mark is set as “GCTAGC.” Here, the position of a label inserted in the six-base motif is expressed as “GCTAG\hat{ }C” using “\hat{ }”. The position of the label is determined by Nicking enzyme in an analysis using an actual sample. The nucleotide sequence of a DNA fragment such as “ATGCCCGCTAGCATGCACCAGAATCTAGATGCCACGCTAGCTCCGACAT GCGGCAACCTA” is divided into “ATGCCCGCTAG (11 bases)”, “CATGCACCAGAATCTAGATGCCACGCTAG (29 bases)”, and “CTCCGACATGCGGCAACCTA (20 bases)” by labels. In genome map format, for example, the DNA fragment is expressed, by arranging the number of bases in each section, as “11, 29, 20”. In genome map format in another expression, the DNA fragment is expressed as “0, 12, 41, 61,” such that the appearance position of the label in the six-base motif is expressed as an absolute coordinate (in this example, 12 and 41 (the number of bases from the left end)), and the terminal information (in this example, 0 and 61) is added to the beginning and end to express the total length of the original DNA fragment. Expression in genome map format is not restricted to them. In genome map format, the total length of a reference genome, a DNA fragment, or the like, and the position of a predetermined nucleotide sequence in a reference genome, a DNA fragment, or the like are expressed such that they can be recognized. In genome map format, since bases (A, G, C, and T) in the nucleotide sequence is expressed by position information of a predetermined nucleotide sequence (six-base motif), the amount of information is reduced compared to the original nucleotide sequence.

The processor 102 converts the nucleotide sequence of the reference genome into genome map format by using the four six-base motifs selected in S105 as predetermined nucleotide sequences. The processor 102 stored the converted reference genome in genome map format in the storage unit 106.

In S107, the processor 102 obtains the nucleotide sequences of all DNA fragments that have been obtained from the DNA sequencer 200 and stored in the storage unit 106. The processor 102 converts the obtained nucleotide sequences of all DNA fragments into genome map format by using the four six-base motifs selected in S105 as predetermined nucleotide sequences. The processor 102 stored all of the DNA fragments converted into genome map format in the storage unit 106.

In S108, the processor 102 assembles all of the DNA fragments in genome map format by Whole Genome Map De Novo Assembly to generate assemble contigs in genome map format. In other words, the processor 102 performs alignment by comparing all of the DNA fragments in genome map format, and aligning them such that portions or whole of the DNA fragments that are the same or similar are in the same position. Further, the processor 102 assembles the aligned DNA fragments to generate assemble contigs in genome map format derived from DNA in the sample. The processor 102 stored, in the storage unit 106, the generated assemble contigs in genome map format derived from DNA in the sample. The processor 102 may refer to the reference genome in genome map format stored in the storage unit 106 during the alignment and assembly. Well-known methods for alignment and assembly may be used.

In S109, the processor 102 obtains the assemble contigs in genome map format derived from DNA in the sample which the assemble contigs are stored in the storage unit 106. The processor 102 obtains the reference genome in genome map format stored in the storage unit 106. The processor 102 aligns the assemble contigs in genome map format derived from DNA in the sample to the reference genome in genome map format to detect structural variations (SVs) in the sample DNA. Structural variation is a mutation with a size of 50 base pair (bp) or more, among genomic differences between organisms. Depending on the mutation pattern, the types of structural variation include deletion, a loss of a part of a nucleotide sequence; insertion, an addition of another nucleotide sequence at a particular site; duplication, an addition of a partial region in a duplicated manner; and inversion, a change in the direction of a partial region into the opposite direction. Structural variation is thought to have an effect on various diseases, and thus its detection is regarded as important. Well-known methods for detecting structural variation may be used. The processor 102 stores, in the storage unit 106, the detected structural variation in the sample DNA. The processor 102 utilizes the assemble contigs and the reference genome in genome map format to reduce the computation load during the detection of structural variation.

Although six-base motifs are used herein, n in n-base motif may be a positive even number other than six. S104 may be omitted, and non-palindromic n-base motifs may be selected as predetermined nucleotide sequences to be used in genome map format. In this case, n may be a positive odd number.

The selection of palindromic six-base motifs in S104 may be performed before S101, and thereafter palindromic six-base motifs contained in the reference genome and DNA fragments in the sample may be counted and the deviation may be calculated. When the palindrome selection is previously performed, counting of non-palindromic six-base motifs and calculation of the deviation can be omitted, which results in reduced computation load.

While the genome assembly device 100 performs both conversion into genome map format and detection of structural variation herein, the conversion and detection may be performed by separate devices.

Four six-base motifs are used herein during conversion into genome map format. The six-base motifs to be selected are not limited to four. When DNA fragments in genome map format are assembled, each DNA fragment preferably includes 15 or more six-base motifs. The 15 or more six-base motifs included in each DNA fragment facilitates the assembly. When DNA fragments contain an average of 10 to 20-kb bases, use of four six-base motifs having a sample appearance rate of a predetermined value or more empirically allows each DNA fragment to contain 15 or more six-base motifs. When the six-base motifs contained in the DNA fragments are less than 15, the number of six-base motifs used in the conversion into genome map format may be more than four.

### Palindrome

In this specification, the nucleotide sequence of a DNA fragment or the like is converted into genome map format by using palindromic six-base motifs. When palindromic six-base motifs are used, for example, the nucleotide sequence of a DNA fragment that is converted into genome map format, and the nucleotide sequence of the complementary strand of the DNA fragment that is read in the opposite direction, converted into genome map format, and again read in the opposite direction are the same. DNA fragments may be read in the opposite direction during assembly of the DNA fragments. Thus, use of palindromic six-base motifs have an advantage that an original DNA fragment converted into genome map format and the complementary strand of the original DNA fragment converted into genome map format can be considered as the same during assembly. When non-palindromic six-base motifs are used, in general, a DNA fragment in genome map format and the complementary strand of the DNA fragment in genome map format are not the same even when one of them is read in the opposite direction before or after the conversion.

### Operation and Effect of the Embodiment

The processor 102 of the genome assembly device 100 of the genome analysis system 10 converts the nucleotide sequence of a DNA fragment output from the DNA sequencer 200 into genome map format. In addition, the processor 102 selects a six-base motif to be used in the conversion into genome map format based on the appearance rate, the quality value, and the deviation of the six-base motif. The six-base motifs selected based on the appearance rate, the quality value, and the deviation allow the genome assembly device 100 to produce more accurate assemble contigs. Since six-base motifs to be used in conversion into genome map format are selected based on the nucleotide sequences of DNA fragments output from the DNA sequencer 200, appropriate six-base motifs can be selected according to the method of basecall by the DNA sequencer 200, the species of the organism, or the like. Thus, the genome assembly device 100 can produce appropriate assemble contigs using any outputs obtained from the DNA sequencer 200. The processor 102 assembles the DNA fragments converted into genome map format to produce assemble contigs. The processor 102 detects a structural variation from the sample, based on the produced assemble contigs in genome map format derived from the sample and the reference genome converted into genome map format. The genome assembly device 100 utilizes assemble contigs converted into genome map format, and thereby can detect a structural variation with reduced computation load.

### Computer Readable Storage Medium

A program for allowing a computer or other machine or device (hereinafter, a computer or the like) to implement any of the functions described above can be recorded on a storage medium readable by the computer or the like. Then, the function can be provided by allowing the computer or the like to read and execute the program in the storage medium.

As used herein, the storage medium readable by a computer or the like refers to a storage medium which can store information such as data and programs through electrical, magnetic, optical, mechanical, or chemical action, and from which a computer or the like can read such information. Such storage medium may be provided therein with computer components such as a CPU and a memory, and the CPU may execute a program.

Examples of the storage medium which is removable from the computer or the like include a flexible disk, a magneto-optical disk, a CD-ROM, a CD-R/W, a DVD, a DAT, an 8 mm tape, and a memory card.

Examples of the storage medium which is fixed to the computer or the like include a hard disk and a ROM.

### Others

These above-described embodiments of the present invention are only illustrative, and the present invention is not limited thereto. Without departing from the spirit and scope of the claims, various modifications, including combinations of components, may be made within the knowledge of those skilled in the art.

### DESCRIPTION OF SYMBOLS

10 Genome analysis system

100 Genome assembly device

102 Processor

104 Memory

106 Storage unit

108 Communication unit

110 Input/output unit

200 DNA sequencer

1. A method of assembling a genome, the method comprising allowing a computer to execute the following steps:
determining the reference appearance rates, that are the appearance rates of all n-base motifs in the nucleotide sequence of a reference genome, wherein the n-base motif is a nucleotide sequence comprising n (n is a predetermined natural number) bases, and wherein the reference genome is the standard genome of an organism; and the sample appearance rates, that are the appearance rates of all of the n-base motifs in the nucleotide sequences of DNA fragments, wherein the DNA fragments is based on DNA extracted from a sample derived from the organism;
calculating the deviations of the sample appearance rates from the reference appearance rates for all of the n-base motifs;
selecting a predetermined number of n-base motifs with smallest deviations, wherein the n-base motifs each have a sample appearance rate of not less than a predetermined value;
converting the nucleotide sequences of the DNA fragments into DNA fragments in genome map format using the predetermined number of n-base motifs selected; and
assembling the DNA fragments converted in genome map format to generate assemble contigs derived from the DNA in the sample.

2. The method of assembling a genome according to claim 1,
wherein n is a predetermined positive even number; and
wherein, when the predetermined number of n-base motifs are selected, the n-base motifs that are palindromic are selected.

3. The method of assembling a genome according to claim 1,
wherein, when the predetermined number of n-base motifs are selected, the n-base motifs that have a quality value of the predetermined value or more are selected.

4. The method of assembling a genome according to claim 1, the method comprising the following steps of:
converting the nucleotide sequence of the reference genome into a reference genome in genome map format using the predetermined number of n-base motifs selected; and
detecting structural variations in the DNA in the sample based on the reference genome in genome map format and the assemble contig.

5. The method of assembling a genome according to claim 1,
wherein the deviation is calculated as an absolute value of (1−(sample appearance rate/reference appearance rate)).

6. A non-transitory computer readable medium for storing a program, wherein the program allows a computer to execute the following steps of:
determining the reference appearance rates, that are the appearance rates of all n-base motifs in the nucleotide sequence of a reference genome, wherein the n-base motif is a nucleotide sequence comprising n (n is a predetermined natural number) bases, and wherein the reference genome is the standard genome of an organism; and the sample appearance rates, that are the appearance rates of all of the n-base motifs in the nucleotide sequences of DNA fragments, wherein the DNA fragments is based on DNA extracted from a sample derived from the organism;
calculating the deviations of the sample appearance rates from the reference appearance rates for all of the n-base motifs;
selecting a predetermined number of n-base motifs with smallest deviations, wherein the n-base motifs each have a sample appearance rate of not less than a predetermined value;
converting the nucleotide sequences of the DNA fragments into DNA fragments in genome map format using the predetermined number of n-base motifs selected; and
assembling the DNA fragments converted in genome map format to generate assemble contigs derived from the DNA in the sample.

7. A genome assembly device, comprising
a storage unit for storing the nucleotide sequence of a reference genome that is the standard genome of an organism, and the nucleotide sequence of a DNA fragment based on DNA extracted from a sample from the organism; and
a processor for executing the following steps of:
determining the reference appearance rates, that are the appearance rates of all n-base motifs in the nucleotide sequence of a reference genome, wherein the n-base motif is a nucleotide sequence comprising n (n is a predetermined natural number) bases; and the sample appearance rates, that are the appearance rates of all of the n-base motifs in the nucleotide sequences of DNA fragments;
calculating the deviations of the sample appearance rates from the reference appearance rates for all of the n-base motifs;
selecting a predetermined number of n-base motifs with smallest deviations, wherein the n-base motifs each have a sample appearance rate of not less than a predetermined value;
converting the nucleotide sequences of the DNA fragments into DNA fragments in genome map format using the predetermined number of n-base motifs selected; and
assembling the DNA fragments converted in genome map format to generate assemble contigs derived from the DNA in the sample.
